(19) United States
(12) Patent Application Publication
(10) Pub. No.: US 2025/0058040 A1
(43) Pub. Date: Feb. 20, 2025

(54) CASSETTE WITH PRESS-FIT FREE-FLOW PREVENTION FOR INFUSION PUMP

(71) Applicant: B. BRAUN MEDICAL INC., BETHLEHEM, PA (US)

(72) Inventors: Michael Huk, Lehighton, PA (US); Linda Ammermann, Mount Bethel, PA (US); Shawn Zigmont, Easton, PA (US); Michael Janders, Northampton, PA (US)

(73) Assignee: B. BRAUN MEDICAL INC., BETHLEHEM, PA (US)

(21) Appl. No.: 18/235,654

(22) Filed: Aug. 18, 2023

(51) Int. Cl. A61M 5/142

(52) U.S. Cl. CPC A61M 5/142; A61M 2205/12; A61M 2205/3331

(57) ABSTRACT

A cassette with free-flow prevention for use with an ambulatory pump. The cassette includes a free-flow prevention device and a housing. The housing is configured to receive the free-flow prevention device through press-fit insertion to facilitate manufacturing. The housing includes an external face, a press plate coupled to the external face, a lever arm retention system to retain the free-flow prevention device, and vertical sidewalls extending from the external face. The housing additionally includes cutouts in the external face adjacent the vertical sidewalls that permit outward bending of the vertical side walls during press-fit insertion of the free-flow prevention device in a direction perpendicular to the press plate.

### FIELD OF THE INVENTION

The present disclosure is related to infusion pumps and, more particularly, to a cassette for use with an infusion pump that includes a press-fit free-flow prevention device.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use. This is beneficial for patients who would otherwise be confined to a bed, and it can help patients get some light exercise by walking or stretching. This also allows fluids to be delivered while patients are being transferred.

There are two conventional types of infusion pumps, syringe pumps that depress a syringe to deliver fluid from the syringe to a patient, and peristaltic pumps that act on a tube to control the rate of fluid flow through the tube from a bottle or bag of fluid to a patient. A concern with peristaltic pumps utilizing tubing is that the force of gravity may cause an unintentional flow of fluid from the bottle or bag of fluid through the tube to the patient when the peristaltic pump is not engaged with the tube, which is commonly referred to as free-flow.

### SUMMARY

Examples described herein are directed to a cassette for use with an ambulatory pump. The cassette includes a free-flow prevention device and a housing. The housing is configured to receive the free-flow prevention device through press-fit insertion to facilitate manufacturing. The free-flow prevention device includes a clamping section having a first side and a second side, a first elongate section extending from the first side, and a second elongate section extending from the second side (forming a U-shaped clamp). The housing supports the free-flow prevention device and defines a channel for receiving a tube. The elongate sections extend parallel to the channel and the clamping section extends across the channel.

The housing includes an external face, a press plate coupled to the external face, a lever arm retention system configured to retain the free-flow prevention device when received, the lever arm retention system including a first lever arm configured to engage the first elongate section and a second lever arm configured to engage the second elongate section, and vertical sidewalls extending from the external face. Other structures, such as ramps on the vertical sidewalls, may be configured and positioned within the cassette to engage and retain the elongate sections. The vertical sidewalls include a first vertical sidewall spaced from the first lever arm and a second vertical sidewall spaced from the second lever arm. The housing additionally includes cutouts in the external face adjacent the first and second vertical sidewalls and the first and second lever arms that permit outward bending of the first and second vertical side walls and potentially inward bending of the first and second lever arms during press-fit insertion of the free-flow prevention device in a direction perpendicular to the press plate.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1A is a perspective view of an example ambulatory infusion pump.

FIG. 1B is a perspective view of an example cassette with a free-flow prevention clamp for use with the ambulatory pump of FIG. 1A.

FIG. 1C is a cutaway perspective view illustrating a cross-section of the sidewall of the cassette of FIG. 1B.

FIG. 1D is a cutaway perspective view illustrating a portion of another example cassette for use with the ambulatory pump of FIG. 1A.

FIG. 2A is a partial perspective view of the cassette of FIG. 1B illustrating the free-flow prevention device in an open, pre-operational state (e.g., shipped state).

FIG. 2B is a partial perspective view of the cassette of FIG. 1B illustrating the free-flow prevention device in a closed, post-operational state.

FIG. 2C is a partial perspective view of the pump of FIG. 1A illustrating arc cams that engage the free-flow prevention device when the cassette is coupled to the pump.

FIG. 2D is a partial perspective view of the pump of FIG. 1A illustrating wedge cams that engage the free-flow prevention device to shift the free-flow prevention device from a shipped state to an operational state when the cassette is coupled to the pump.

FIG. 3A is a partial side view of the example cassette of FIG. 1B depicting the free-flow prevention device in an open condition and a closed condition.

FIG. 3B is a partial side view of the example cassette of FIG. 1B illustrating features of the cassette for configuring the free-flow prevention device in an open pre-operational state, an open operational state, and a closed post-operational state.

FIG. 4A is a top view of a cassette configured to receive a free-flow prevention device.

FIG. 4B is a bottom partial view of the cassette of FIG. 4A.

FIG. 4C is a perspective end cut away view of the cassette of FIG. 4A.

FIG. 4D is a perspective side cut away view of the cassette of FIG. 4A illustrating a lever arm.

FIG. 4E is a side partial cut away view of the cassette of FIG. 4A illustrating a positioning hook.

FIG. 4F is a side partial cut away view of the cassette of FIG. 4A illustrating positioning of the free-flow prevention device within the cassette.

FIG. 5 is a flow chart of example steps for press-fit insertion of a free-flow prevention device.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

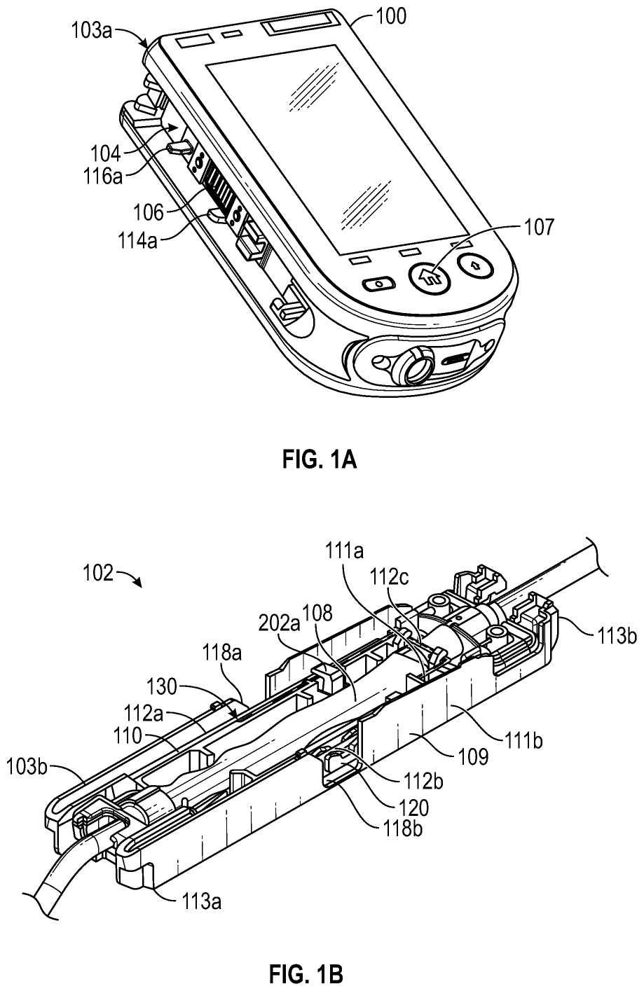

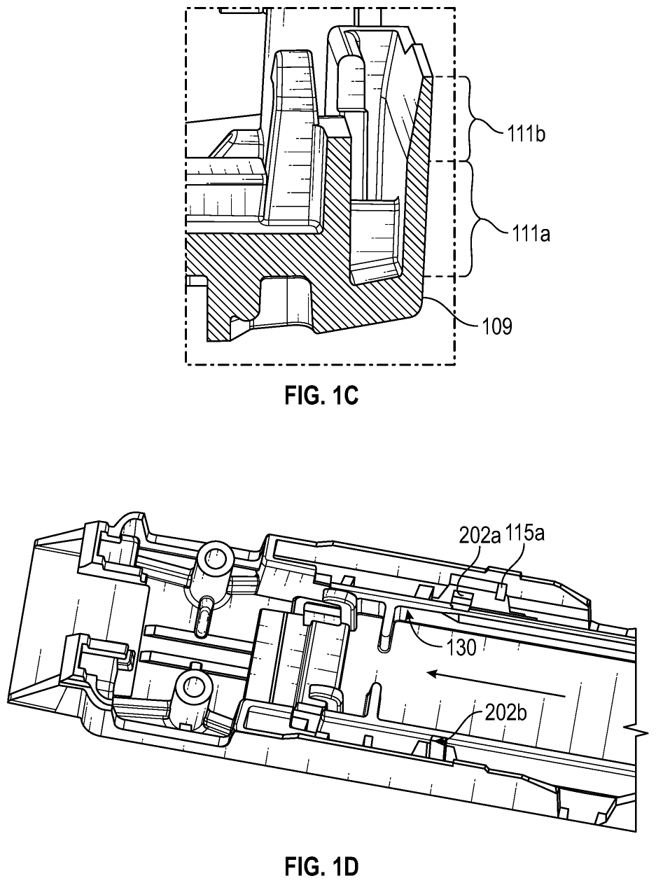

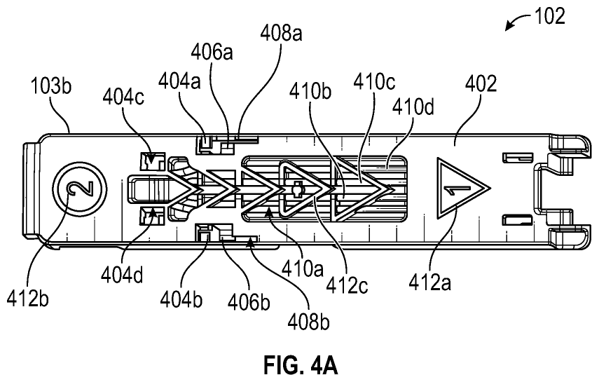

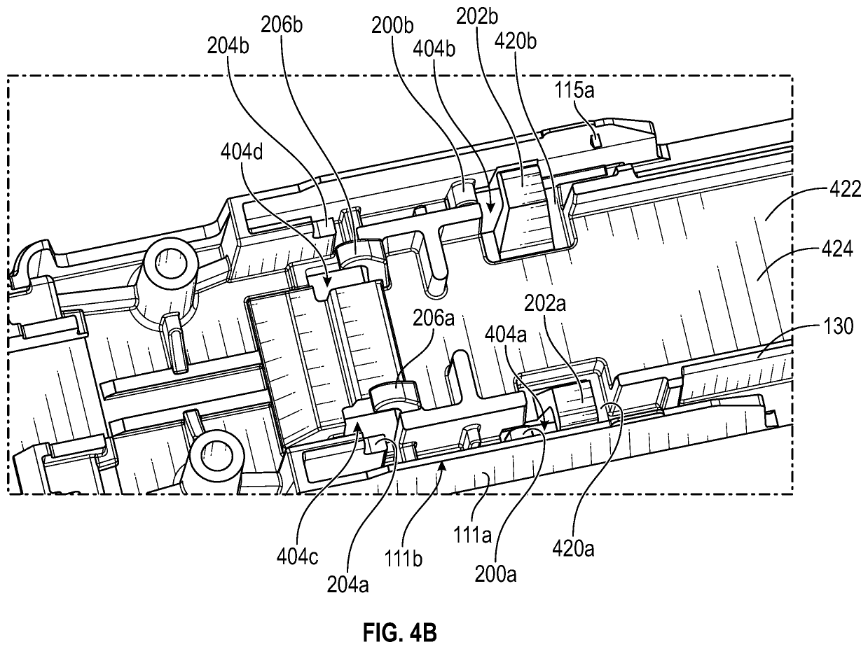

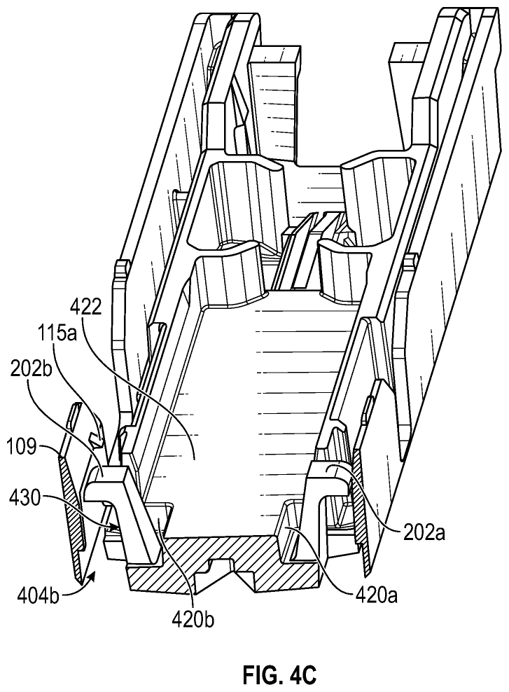

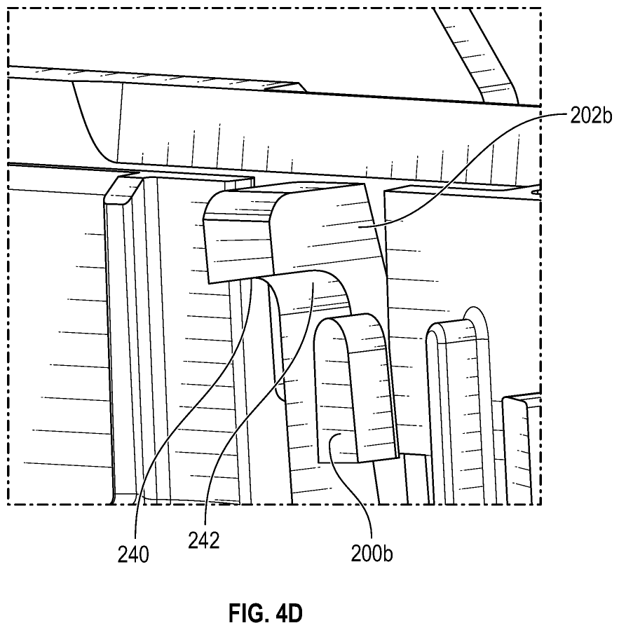

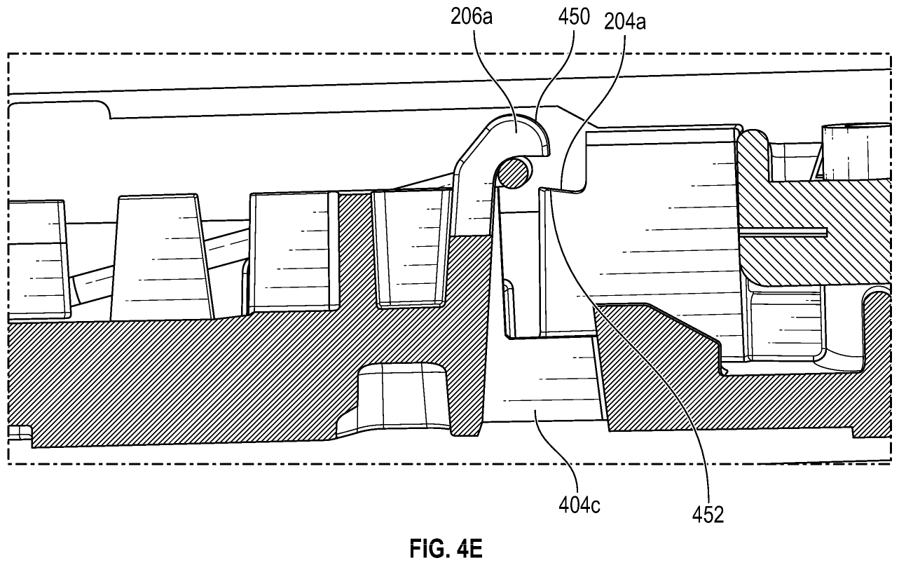

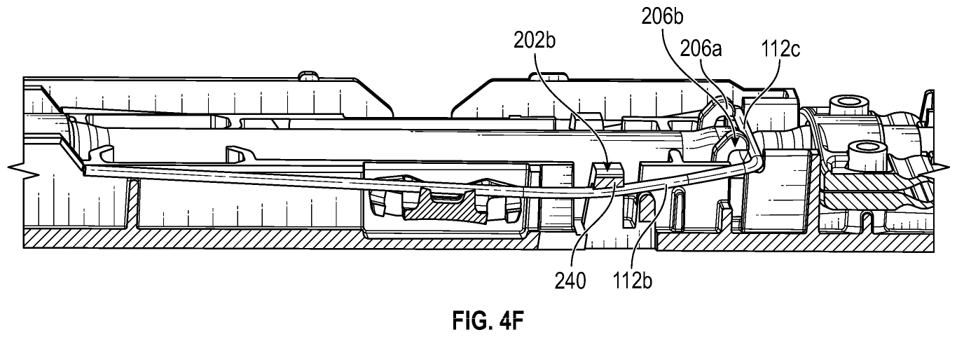

FIG. 1A depicts an example ambulatory pump 100 and FIG. 1B depicts an example cassette 102 with free-flow prevention for use with the ambulatory pump 100. The ambulatory pump 100 includes a receptacle 104 configured to receive the cassette 102. A peristaltic pump 106 within the receptacle 104 acts upon a tube 108 extending through a channel 424 (FIG. 4B) within the cassette 102 to pump fluid from a fluid container (e.g., a bag or a bottle; not shown) into a patient. Channel walls (e.g., channel wall 130) within the cassette 102 partially define the channel 424. In one example, the channel walls 130 may include breaks or cutouts as illustrated in FIG. 1B (e.g., for lever arms 202a, 202b; described in further detail below). In other examples, the channel walls 130 may be continuous as illustrated in FIG. 1D (with, for example, the lever arms 202a, 202b integrated into the side walls 130).

An example free-flow prevention device 110 is positioned within the cassette 102 to allow fluid flow through the tube 108 when the cassette is coupled to the ambulatory pump 100 within the receptacle 104. When the cassette 102 is coupled to the pump, the pump 100 raises the free-flow prevention device 110 and the peristaltic pump 106 controls fluid flow through the tube 108. When the cassette 102 is subsequently disengaged from the pump, the free-flow prevention device 110 cuts off fluid flow through the tube 108 in order to prevent unintentional fluid flow through the tube (e.g., free-flow).

The ambulatory pump 100 includes a user interface for interacting with the ambulatory pump 100. The illustrated user interface includes a display (which may be a touchscreen) and buttons 107. A user controls operation of the ambulatory pump via the user interface. The pump 100 additionally includes a housing 103a containing and supporting the components of the ambulatory pump 100 such as the peristaltic pump 106, electronics, and power supplies.

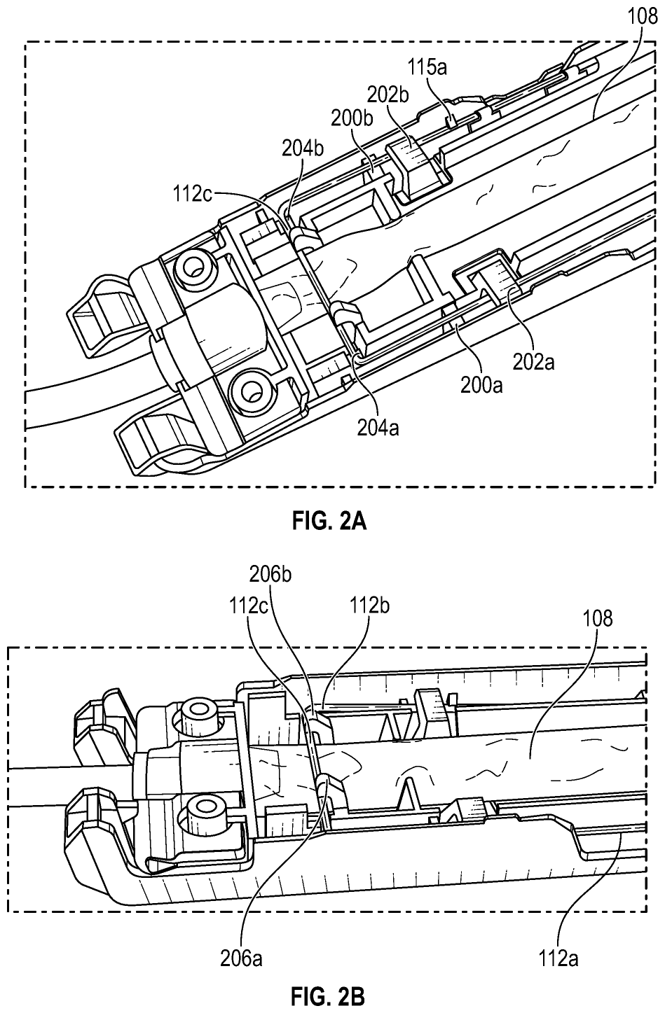

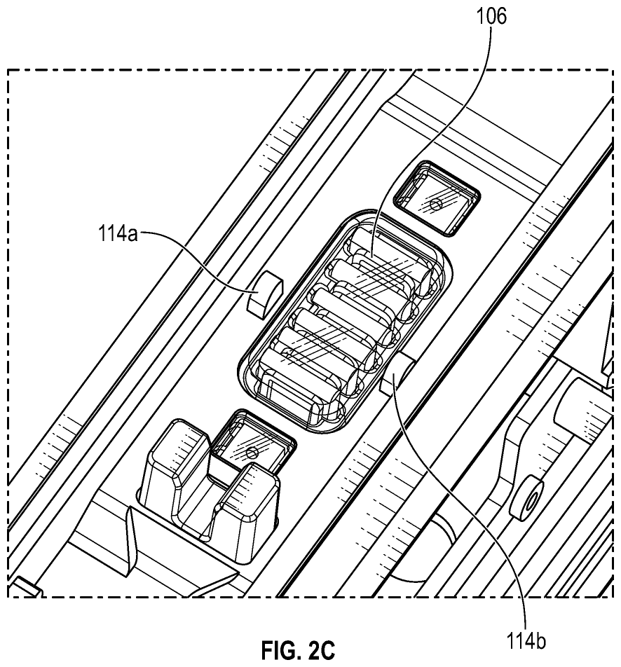

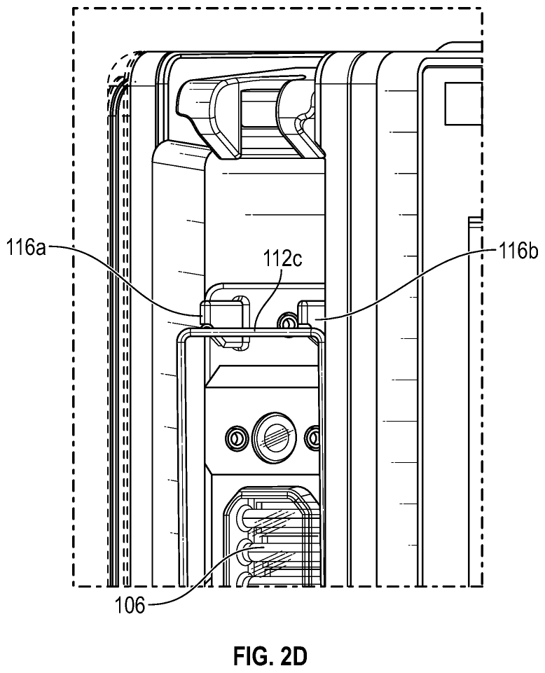

The free-flow prevention device 110 includes a first elongate section 112a, a second elongate section 112b, and a clamping section 112c (forming a U-shaped clamp). The housing 103b of the cassette 102 supports the free-flow prevention device 110. The housing includes multiple lower supports (e.g., override fulcrums 200a and 200b; FIG. 2A). The clamping section 112c is positioned within the cassette geometry such that, when the cassette 102 is received within the receptacle 104 of the ambulatory pump 100, the clamping section 112c extends across the channel receiving the tube 108. The housing of the cassette 102 may be rigid plastic or other material capable of supporting the tube 108 and free-flow prevention device 110.

The first elongate section 112a extends from a first side of the clamping section 112c and along a first side of the cassette 102 parallel to the channel within the cassette 102 receiving the tube 108. The second elongate section 112b extends from a second side of the clamping section 112c and along a second side of the cassette 102 parallel to the channel receiving the tube 108. In the illustrated example, the free-flow prevention device 110 is a staple-shaped leaf spring consisting of single piece of metal (e.g., spring steel). In other examples, the free-flow prevention device 110 may be formed in pieces and/or of different materials (e.g., the elongate sections 112a, b may be metal and the clamping section 112c may be rigid plastic).

The ambulatory pump 100 also includes a pair of arc cams (a first arc cam 114a on one side of the receptacle is illustrated FIG. 1A, with the second hidden from view) for engaging the elongate sections 112a, b of the free-flow prevention device in order to lift the clamping section 112c. Additionally, the ambulatory pump 100 includes a pair of wedge cams (a first wedge cam 116a on one side of the receptacle 104 is illustrated FIG. 1A, with the second hidden from view) for transitioning the free-flow prevention device 110 from an open, manufactured/shipped state to an operational state, which is described in further detail below.

The cassette 102 also includes a first cutout 118a in a vertical sidewall 109 of the cassette 102 and a second cutout 118b in an opposite sidewall of the cassette 102. Additionally, the cassette 102 includes a touch pad 120 positioned on an elongate section 112b adjacent a midpoint of the elongate section 112b and the cutout 118b. The touch pad 120 and cutout 118b facilitates engagement of the second elongate section 112b by a finger of an operator in order to manually lift the clamping section 112c to allow fluid flow through the tube 108 (e.g., for priming the cassette 102) when the cassette 102 is not received within the receptacle 104 of the ambulatory pump 100. The touch pad 120 may be a press-fit piece of rigid plastic. Although the touch pad 120 is illustrated as only on one elongate section 112b, a touch pad may also be provided on the other elongate section 112a. Additionally, the touch pad 120 may be omitted and an operator may engage the elongate sections 112a, b directly to manually lift the clamping section 112c.

As illustrated in FIG. 1C, the vertical side walls 109 include a first region 111a of substantially uniform thickness (e.g., 1.0 mm) and a second region 111b that is chamfered (e.g., narrowing from 1.0 mm proximate the first region 111a to 0.48 mm near an edge of the cassette 102). The first and second regions 111 of the sidewalls 109 are configured to allow outward bending of the respective sidewall during insertion of the free-flow prevention device 110 to facilitate manufacturing.

FIG. 2A depicts the cassette 102 with the free-flow prevention device 110 in an open, pre-operational state (e.g., manufactured/shipped state). The cassette 102 includes a first ledge 204a adjacent a first side of the clamping section 112c on one side of the channel receiving the tube 108 and a second ledge 204b adjacent a second side of the clamping section 112a on an opposite side of the channel receiving the tube 108. The ledges 204a, b support the clamping section 112c and oppose the downward force being applied to the clamping section 112c by the first and second elongate sections 112a, b. This prevents the clamping section 112c from occluding the tube 108 (e.g., for Ethylene Oxide, EtO, sterilization and/or priming) prior to insertion of the cassette 102 into the ambulatory pump 100.

The housing of the cassette 102 additionally includes a pair of override fulcrums 200a, b positioned below respective elongate sections 112a, b of the free-flow prevention device 110 and a pair of lever arms 202a, b forming a lever arm retention system positioned above respective elongate sections 112a, b of the free-flow prevention device 110. The override fulcrums 200a, b are positioned between a midpoint of the first and second elongate sections 112a, b and the clamping section 112c. The lever arms 202a, b are positioned between the midpoint of the first and second elongate sections 112a, b and the override fulcrums 200a. The housing may include addition structures such as a ramp(s) 115a on the vertical sidewalls, which may be configured and positioned within the cassette to engage and retain the elongate sections.

After the free-flow prevention device 110 is moved off the ledges 204 and into an operational state, applying a force to the midpoints of the first and second elongate sections 112a, b (e.g., by the arc cams 114a, b upon attachment of the cassette 102 to the receptacle 104 of the ambulatory pump or by a user's finger) pivots the elongate sections 112a, 112b about the override fulcrums 200a, b to raise the clamping section 112c and allow fluid flow through the tube 108. Removing the force while in this state (e.g., by detaching the cassette or withdrawing the user's finger) shifts the pivot to occlusion fulcrums on the lower side of the lever arms 202a, b, which lowers the clamping section 112c and blocks fluid flow through the tube 108.

FIG. 2B depicts the cassette 102 with the free-flow prevention device 110 in a closed, operational state and FIG. 2C depicts the arc cams 114 and peristaltic pump 106 of the ambulatory pump 100. Positioning hooks 206a, b are provided to facilitate placement of the clamping section 112c of the free-flow prevention device 110 onto the ledges 204, which maintains the tube 108 in an open condition. FIG. 2D depicts the wedge cams 116a, b and their relationship to the free-flow prevention device when the cassette (not shown in FIG. 2D) is attached to the ambulatory pump 100.

The free-flow prevention device 110 is moved off the ledges 204 and out of the open, pre-operational state by the wedge cams 116 as the cassette 102 is attached to the receptacle 104 of the ambulatory pump 100. To attach the cassette 102 to the receptacle 104, a user first inserts an end 113a of the cassette 102 into the receptacle 104 near a bottom of the ambulatory pump 100 (also referred to as a toe-in). The user then pushes the opposite end 113b of the cassette 102 downward such that the cassette 102 rotates about the toe-in into full engagement with the ambulatory pump 100. As the cassette 102 is rotated into position, the arc cams 114 engage the elongate sections 112a, b to apply a force that raises the clamping section 112c and angled portions of the wedge cams 116 substantially simultaneously engage the clamping section 112c to shift the free-flow prevention device 110 away from the ledges 204. In this manner, the tube 108 remains unobstructed by the clamping section 112c when the cassette 102 is attached to the ambulatory pump 100, allowing the peristaltic pump 106 to control fluid flow through the tube 108. In an example, once the free-flow prevention device 110 is shifted from the shipped/pre-operational state into the operational state by the wedge cams 116, the free-flow prevention device 110 remains in the operational state.

Removal of the cassette 102 from the receptacle 104 is accomplished by reversing the process. As the cassette 102 is rotated out of engagement with the ambulatory pump 100, the arc cams 114 disengage from the elongate sections 112a, b. With the force from the arc cams 114 removed, and the ledges 204 no longer positioned under the clamping section 112c due to the shift of the free-flow clamp 110 during the insertion of the cassette 102, the clamping section 112 rotates downward about the occlusion fulcrums on the lower side of the lever arms 202a to close off the tube 108 as depicted in FIG. 2B.

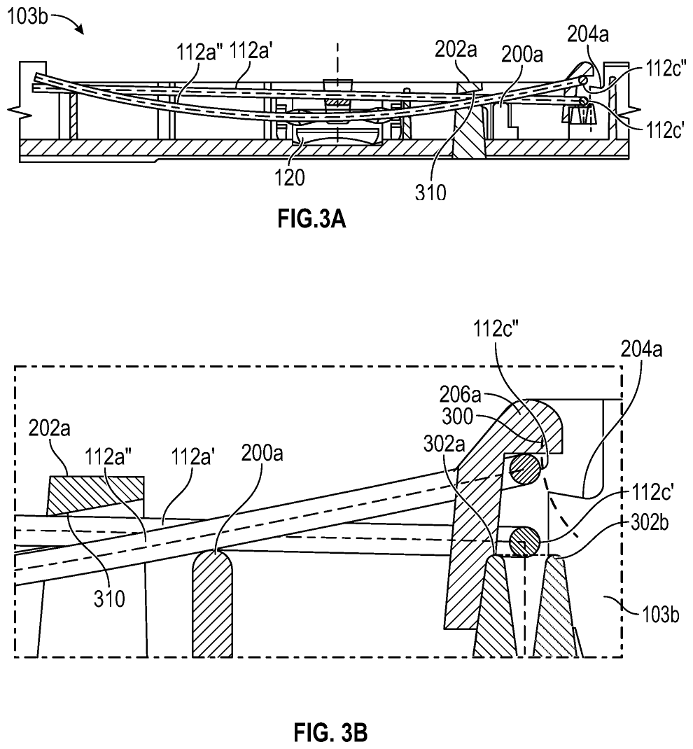

FIG. 3A depicts movement of an elongate section 112 after shifting the free-flow prevention device 110 off the ledge 204a from the open, pre-operational state into the operational state. In the operational state, the first elongate section 112a can be in a closed condition (represented by first elongate section 112a′) or an open condition (represented by first elongate section 112a″). In the closed condition, no force is applied to the midpoint of the first elongate section 112′, resulting in the clamping section 112c rotating downward about an occlusion fulcrum 310 on the lower surface of the lever arm 202a to close off the tube 108. In the open condition, force is applied to the midpoint of the first elongate section 112″, resulting in the clamping section 112c rotating upward about the override fulcrum 200a to open the tube 108.

FIG. 3A additionally depicts movement of the touch pad 120 responsive to force applied at the midpoint of the first elongate section 112. In the absence of applied force, touch pad 120 is in a closed position that maintains clamping section 112c′ clamped against the tube 108. When force is applied (e.g., by an arc cam 114a or a user's finger), touch pad 120 is in an open position that raises clamping section 112c″ to permit fluid flow through the tube 108.

FIG. 3B depicts geometry of a portion of the housing 103b of the cassette 102 that supports the free-flow clamp 110. Shifting of the clamping section 112c of the free-flow clamp 110 is represented by dashed wedge cam shift line 300. Occlusion fulcrum 310 on lower surface of lever arm 202a acts on elongate section 112a′ forcing clamping section 112c′ downward to occlude the tube 108. In the illustrated example, occlusion ribs 302a and 302b are positioned perpendicular to the channel receiving the tube 108 and on either side of the clamping section 112c to assist with complete closure of the tube 108 when the clamping section 112c is in the closed condition. In an alternative example, the occlusion ribs 302a and 302b are replaced by a single flat section wherein the tube 108 is positioned between the clamping section 112c and the flat section. Override fulcrum 200a acts on elongate section 112a″ forcing clamping section 112c″ upward to enable fluid flow through the tube 108.

Examples of the free-flow prevention device 110 enable the cassette 102 to maintain the clamping section 112c in an un-occluded/open state prior to a first attachment of the cassette to the receptacle, maintain the clamping section in the un-occluded/open state upon the first attachment of the cassette to the receptacle, and transition the clamping section to an occluded/closed state upon detachment of the cassette from the receptacle. Additionally, examples of the free-flow prevention device 110 enable the cassette to manually transition the clamping section to an un-occluded state after detachment of the cassette from the receptacle and/or transition the clamping section to the un-occluded state upon subsequent attachments of the cassette to the receptacle.

FIG. 4A depicts a top view of a cassette housing 103b (i.e., the surface facing away from the pump 100 when inserted). The housing 103b includes an external face 402 that faces away from the ambulatory pump 100 when the cassette 102 is inserted into the ambulatory pump 100. The external face 402 includes multiple cutouts 404, multiple ribs (4 ribs 410a-d) for maintaining rigidity of the press plate 422 (FIG. 4B), insertion indicia 412a and 412b, and flow direction indicia 412c. The insertion indicia 412 includes a first insertion indicia 412a (the numeral “1” in a triangle indicating a first step of insertion; e.g., toe-in of one end of the cassette 102 within the pump 100; and also indicating direction of insertion, which matches flow direction in this implementation) and a second insertion indicia 412b (the numeral “2” in a circle indicating a second step of insertion; e.g., press and lock of the other end of the cassette 102 within the pump 100). The flow direction indicia 412c indicates direction of fluid flow with a series of triangles formed in the external face 402 of the cassette housing 103b.

As described in further detail below, two cutout (cutouts 404a and 404b) correspond to respective lever arms 202a and 202b and two cutouts (cutouts 404c and 404d) correspond to respective positioning hooks 206a and 206b. The cutouts are sized and positioned to permit flexibility of the lever arms 202 and vertical side walls 109 during press-fit insertion of the free-flow prevention device 110, while retaining sufficient structural integrity of the cassette 102 for forcing fluid through a tube 108 within the cassette 102 using the peristaltic pump 106 of the ambulatory pump 100. In particular, the cutouts 404 in the walls of the cassette 102 permit inward movement of the lever arms 202 and outward bending of the vertical side walls 109 in the vicinity of the lever arms during press-fit insertion of the free-flow prevention device 110. In the illustrated example, cutouts 404c and 404d are rectangular and cutouts 404a and 404b each include a respective first rectangular region 406a and 406b and a respective second, smaller rectangular region 408a and 408b.

FIGS. 4B and 4C depicts a bottom view of the cassette 102 (i.e., the surface facing the pump 100 when inserted) in accordance with one example. The cassette 102 includes a press plate 422 that is coupled to the external face 402 (e.g., via ribs 410; FIG. 4A). The press plate 422 includes a channel 424 for receiving the tubing 108 (FIG. 1B). The press plate 422 along with the sidewalls 130 extending therefrom at least partially define the channel 424.

In the illustrated example, the cassette 102 includes a first well 420a in the press plate 422 surrounding the first lever arm 202a and a second well 420b in the press plate 422 surrounding the second lever arm 202b. The wells 420 facilitate inward bending of the respective lever arms 202 (in addition to bending permitted by the respective cutouts 404). In an alternative example, the first and second wells 420a and 420b may be omitted with the lever arms 202a and 202b integrated into the side walls 130 (see FIG. 2D). The size and location of the cutouts and wells, the thickness and chamfering of the vertical side walls 111, and the materials used can be adjusted to alter the insertion force needed to press-fit insert the free-flow prevention device 110 to facilitate manufacturing. It is contemplated that one or more of the cutouts 404, wells 420, or chamfering may be omitted if suitable insertion forces for the free-flow prevention device 110 are achieved with the remaining structures. In an example, the insertion force is between 100 and 400 newtons (e.g., between 140 newtons and 200 newtons).

FIG. 4C depicts a cut-away end view of the cassette 102 at the lever arms 202. The second lever arm 202b includes an angled extension 430 extending from the well 420b toward a vertical sidewall 109. The first lever arm 202a also includes an angled extension that is a mirror image of the angled extension 430. The angled extensions further permits inward bending of the respective lever arms 202 during press-fit insertion of the free-flow prevention device 110. In an example, the angled extensions have an angle of between 5 degrees and 20 degrees from perpendicular to the press plate 422.

FIG. 4D depicts a cut-away perspective view of the second lever arm 202b and a fulcrum 200b. The lever arm 202b includes an angled portion 240 that acts as an upper pivot point and the fulcrum 200b acts as a lower pivot point/support to permit rotational movement of the free-flow prevention device 110 (e.g., during insertion of the cassette 102 into the pump 100 or manual manipulation by an operator). The angled portion 240 additionally reduces stress on the free-flow prevention device during movement and distributes force across the face of the angled portion 240. In an example, the angled portion 240 has an angle of between 5 degrees and 20 degrees with respect to the press plate 422. The second lever arm 202b additionally includes a curved portion 242 between the angled extension 430 (FIG. 4C) and the angled portion to further reduce stress on the free-flow prevention device and the lever arms 202 202a, 202b during movement. The first lever arm 202a is a mirror image of the second lever arm 202b.

FIG. 4E depicts a cut-away side view of the first positioning hook 206a. The first positioning hook 206a includes a curved guiding surface 450 to facilitate positioning of the free-flow prevention device 110 on the ledge 204a. Additionally, the ledge 204a has an angled surface 452 to facilitate positioning of the free-flow prevention device 110 on the ledge 204a and retaining the free-flow prevention device on the ledge until it is deliberately moved off the ledge by the wedge cams 116 (FIG. 2D). The cutout 404c adjacent the first hook 206a enables a mold tool to form the underside of the positioning hook and optionally may be configured to permit flexing of the positioning hook away from the ledge 204a during insertion of the free-flow prevention device. The second positioning hook 206b is the same or substantially similar to the first positioning hook 206a in construction and operation.

FIG. 4F depicts a cutaway perspective view of the cassette with the free-flow prevention device 110 installed and in an open position prior to movement off the ledges 204 (e.g., in a shipping state). In this state, the second elongate portion 112b abuts the angled surface 240 of the second lever arm 202b (FIG. 4D), the first elongate portion 112a abuts the angled surface of the first lever arm (not shown), and the clamping section 112c is supported by the ledge 204 (FIGS. 2A and 4E) enabling fluid to flow through the tubing 108.

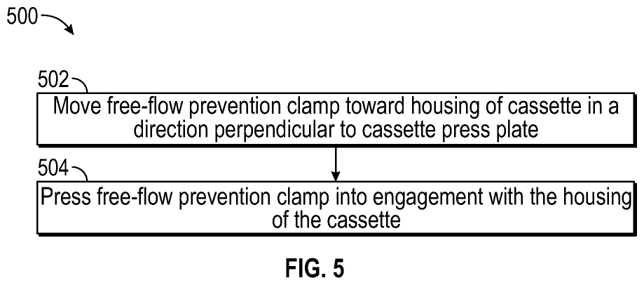

FIG. 5 depicts a flowchart 500 of example steps of a manufacturing process for press-fit insertion of a free-flow prevention device into a housing of a cassette. Although the steps are described with reference to cassette 102 and free-flow prevention device 110, other suitable cassettes and free-flow prevention devices in which one or more steps of the flowchart 500 can be practiced will be understood by one of skill in the art from the description herein. Additionally, it is contemplated that one or more of the steps shown in FIG. 5, and described herein, may be omitted, performed simultaneously or in series, performed in an order other than illustrated and described, or performed in conjunction with additional steps.

At block 502, move the free-flow prevention device 110 toward a housing 103b of the cassette 102. In an example, the free-flow prevention device 110 is moved in a direction perpendicular to the press plate 422 of the cassette housing 103b. The free-flow prevention device may be moved manually or using production apparatus. The production apparatus may be a robotic assembly system including a support configured to support the cassette housing 103b in a predefined orientation and a robotic arm configured to grasp, manipulate, and move the free-flow prevention device 110 toward the cassette housing 103b. A processor of the robotic assembly system is programmed with instructions for moving the robotic arm relative to the support in order to move the free-flow prevention device 110 toward the cassette housing 103b. Other suitable production apparatus will be understood by one of skill in the art from the description herein.

At block 504, press the free-flow prevention device 110 into engagement with the housing 103b of the cassette 102. The free-flow prevention device may be pressed into engagement manually or using production apparatus. Wherein production apparatus is used, the processor of the robotic assembly system is further programmed with instructions for moving the robotic arm such that the free-flow prevention device 110 is moved into engagement with the housing with a predefined insertion force that overcomes resistance encountered by the free-flow prevention device due to one or more of the lever arms, vertical sidewalls, and positioning hooks.

In an example, the housing 103b includes an external face 402, a press plate 422 coupled to the external face 402 (e.g., by ribs 410), a first lever arm 202a spaced from a first vertical sidewall 109 extending from the external face 402, and a second lever arm 202b spaced from a second vertical sidewall 109 extending from the external face 402. The first elongate section 112a contacts the first lever arm 202a and the first vertical sidewall 109 and the second elongate section 112b contacts the second lever arm 202b and the second sidewall 109 as the free-flow prevention device 110 is pressed into engagement with the housing 103b. The housing 103b includes cutouts 404 in the external face 402 adjacent the first and second vertical sidewalls 109 and the first and second lever arms 202 that permit outward bending of the first and second vertical side walls and inward movement of the first and second lever arms due to the cutouts in the walls of the cassette 102 to allow the respective first and second elongate sections to pass while pressing the free-flow prevention device into engagement with the housing.

The terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A cassette with free-flow prevention that is insertable into an infusion pump having a peristaltic actuator, the cassette comprising:
a free-flow prevention device including a clamping section having a first side and a second side, a first elongate section extending from the first side, and a second elongate section extending from the second side; and
a housing configured to receive and support the free-flow prevention device, the housing defining a channel for receiving a tube, the first elongate section and the second elongate section of the free-flow prevention device, when received, extending parallel to the channel and the clamping section extending across the channel, the housing comprising:
an external face;
a press plate coupled to the external face;
a lever arm retention system configured to retain the free-flow prevention device when received, the lever arm retention system including a first lever arm configured to engage the first elongate section and a second lever arm configured to engage the second elongate section;
vertical sidewalls extending from the external face, the vertical sidewalls including a first vertical sidewall spaced from the first lever arm and a second vertical sidewall spaced from the second lever arm; and
cutouts in the external face adjacent the first and second vertical sidewalls that permit outward bending of the first and second vertical side walls during press-fit insertion of the free-flow prevention device in a direction perpendicular to the press plate.

2. The cassette of claim 1, further comprising:
a first channel wall extending from the press plate that at least partially defines the channel, the first lever arm integrated into the first channel wall; and
a second channel wall extending from the press plate that at least partially further defines the channel, the second lever arm integrated into the second channel wall.

3. The cassette of claim 1, wherein the free-flow prevention device is press-fit inserted in the direction perpendicular to the press plate with an insertion force of between 100 newtons and 400 newton.

4. The cassette of claim 1, wherein the first vertical sidewall is chamfered in a first region corresponding to the first lever arm and the second vertical sidewall is chamfered in a second region corresponding to the second lever arm.

5. The cassette of claim 1, wherein the first lever arm comprises a first retaining surface facing the first elongate section and the second lever arm comprises a second retaining surface facing the second elongate section to retain the free-flow prevention device after insertion.

6. The cassette of claim 5, further comprising:
a plurality of supports positioned to support the first and second elongate sections when the free-flow prevention device is received;
wherein the first and second retaining surfaces are each angled and curved to permit rotational movement of the respective first and second elongate sections about a respective one of the plurality of supports.

7. The cassette of claim 6, wherein an angle of each of the first and second retaining surfaces is between 5 degrees and 20 degrees with respect to the press plate.

8. The cassette of claim 6, wherein each of the respective one of the plurality of supports is adjacent a respective one of the cutouts.

9. The cassette of claim 1, further comprising:
a first positioning hook and a second positioning hook extending away from the press plate, each positioning hook having a surface configured to engage the clamping section during insertion to assist in positioning the free-flow prevention device.

10. An infusion system, the system comprising:
an infusion pump having a peristaltic actuator, and
the cassette with free-flow prevention of claim 1.

11. A method for manufacturing a cassette with free-flow prevention, the cassette configured for insertion into an infusion pump, the method comprising:
moving a free-flow prevention device toward a housing of the cassette in a direction perpendicular to a press plate of the cassette, the free-flow prevention device including a clamping section having a first side and a second side, a first elongate section extending from the first side, and a second elongate section extending from the second side, and
pressing the free-flow prevention device into engagement with a housing of the cassette, the housing including an external face, an external face coupled to the press plate, a first lever arm spaced from a first vertical sidewall extending from the external face and a second lever arm spaced from a second vertical sidewall extending from the external face, the first elongate section contacting the first lever arm and the first vertical sidewall and the second elongate section contacting the second lever arm and the second sidewall during the pressing into engagement, the housing further including cutouts in the external face adjacent the first and second vertical sidewalls that permit outward bending of the first and second vertical side walls to allow the respective first and second elongate sections to pass while pressing the free-flow prevention device into engagement with the housing.

12. The method of claim 11, the housing further comprising a first channel wall extending from the press plate that at least partially defines the channel, the first lever arm integrated into the first channel wall; and a second channel wall extending from the press plate that at least partially further defines the channel, the second lever arm integrated into the second channel wall.

13. The method of claim 11, wherein the pressing is performed with an insertion force of between 100 newton and 400 newtons.

14. The method of claim 13, wherein the pressing is performed with an insertion force of between 140 newton and 200 newtons.

15. The method of claim 11, wherein the first vertical sidewall is chamfered in a first region corresponding to the first lever arm and the second vertical sidewall is chamfered in a second region corresponding to the second lever arm.

16. The method of claim 11, wherein the first lever arm comprises a first retaining surface facing the first elongate section and the second lever arm comprises a second retaining surface facing the second elongate section to retain the free-flow prevention device after the pressing into engagement.

17. The method of claim 16, the housing further comprising a plurality of supports positioned to support the first and second elongate sections after the pressing into engagement;
wherein the first and second retaining surfaces are each angled and curved to permit rotational movement of the respective first and second elongate sections about a respective one of the plurality of supports.

18. The method of claim 17, wherein an angle of each of the first and second retaining surfaces is between 5 degrees and 20 degrees with respect to the press plate.

19. The method of claim 17, wherein each of the respective one of the plurality of supports is adjacent a respective one of the cutouts.

20. The method of claim 11, the housing further comprising a first positioning hook and a second positioning hook extending away from the press plate, each positioning hook having a surface configured to engage the clamping section during insertion to assist in positioning the free-flow prevention device.
